SEQUENCE LISTING (36 sequences)

SEQ ID NO: 1 - DNA, 23 bp, Homo sapiens
ctgcgaggaacagaagtgcgagg

SEQ ID NO: 2 - DNA, 25 bp, Homo sapiens
ggatggagttgtcggtgtagatgca

SEQ ID NO: 3 - DNA, 25 bp, Homo sapiens
accgtttttttgcaggatccagatg

SEQ ID NO: 4 - DNA, 24 bp, Homo sapiens
gatggtgcaataatccgaggcttg

SEQ ID NO: 5 - DNA, 20 bp, Homo sapiens
cttcggcccagtggacagcg

SEQ ID NO: 6 - DNA, 22 bp, Homo sapiens
cgtgggaaggtagagcttgggc

SEQ ID NO: 7 - DNA, 21 bp, Homo sapiens
cctgagtgtgaccggcgatgc

SEQ ID NO: 8 - DNA, 25 bp, Homo sapiens
agagaattcattcatggccacgagg

SEQ ID NO: 9 - DNA, 25 bp, Homo sapiens
tgtgacatccgtatccagcttcctg

SEQ ID NO: 10 - DNA, 30 bp, Homo sapiens
cattttcatacctgaaggaacgatcaataa

SEQ ID NO: 11 - DNA, 27 bp, Homo sapiens
tttcaatgtttcgctattcaaattggc

SEQ ID NO: 12 - DNA, 30 bp, Homo sapiens
gtttgacatcagatcttctaaatttcctgc

SEQ ID NO: 13 - DNA, 19 bp, Homo sapiens
ccctggaggcggcgagaac

SEQ ID NO: 14 - DNA, 24 bp, Homo sapiens
cctagacgctggctcctcagtagc

SEQ ID NO: 15 - DNA, 22 bp, Homo sapiens
ccgggacttggagaagcactgc

SEQ ID NO: 16 - DNA, 21 bp, Homo sapiens
ggcaccttgcaggcacctttg

SEQ ID NO: 17 - DNA, 25 bp, Homo sapiens
tcccaccttactacatgcggagacc

SEQ ID NO: 18 - DNA, 22 bp, Homo sapiens
taggctgtctttggcgaggagg

SEQ ID NO: 19 - DNA, 27 bp, Homo sapiens
gccctgtggatgactgagtacctgaac

SEQ ID NO: 20 - DNA, 28 bp, Homo sapiens
gccaaactgagcagagtcttcagagaca

SEQ ID NO: 21 - DNA, 24 bp, Homo sapiens
ttaggatccgggagcagcccagag

SEQ ID NO: 22 - DNA, 26 bp, Homo sapiens
ttaagcttgacctctcggggggagtc

SEQ ID NO: 23 - DNA, 25 bp, Homo sapiens
ataggatcctggcttcggggcaagg

SEQ ID NO: 24 - DNA, 32 bp, Homo sapiens
gagaagcttgtactcataggcattctctgccg

SEQ ID NO: 25 - DNA, 31 bp, Homo sapiens
tatggatccagctttcccagaaaggatacag

SEQ ID NO: 26 - DNA, 27 bp, Homo sapiens
cggaagcttgctctgatatgctgtccc

SEQ ID NO: 27 - DNA, 18 bp, Homo sapiens
atccctggccttcatcag

SEQ ID NO: 28 - DNA, 17 bp, Homo sapiens
gcactgctaggccatgg

SEQ ID NO: 29 - DNA, 26 bp, Homo sapiens
agatgtctgtggcttcccttgatctg

SEQ ID NO: 30 - DNA, 25 bp, Homo sapiens
aagtcatcaaagggctcggtcttca

SEQ ID NO: 31 - DNA, 24 bp, Homo sapiens
aacaatgaaaaggcccccaaggta

SEQ ID NO: 32 - DNA, 28 bp, Homo sapiens
tccgtagctgttcaagtttgtgtttcaa

SEQ ID NO: 33 - DNA, 29 bp, Homo sapiens
gacgaatatgaccccacaatagaggattc

SEQ ID NO: 34 - DNA, 29 bp, Homo Sapiens
attattgatggcaaatacacacaggaagc

SEQ ID NO: 35 - DNA, 22 bp, Homo Sapiens
ccaacacagtgctgtctggcgg

SEQ ID NO: 36 - DNA, 26 bp, Homo sapiens
tactcctgcttgctgatccacatctg

(19) United States
(12) Patent Application Publication
(10) Pub. No.: US 2015/0315563 A1
(43) Pub. Date: Nov. 5, 2015

(54) METHOD FOR ACTIVATION OF STEM CELL PROLIFERATION AND INCREASE OF STEM CELLS RESISTENCE TO NEGATIVE IMPACTS

(71) Applicants: Elena Vladimirovna ORLOVA, (US); Evgeny Ilich MAEVSKY, (US); Vladimir Konstantinovich KLUBKOV, (US)

(72) Inventors: Elena Vladimirovna ORLOVA, Moscow (RU); Evgeny Ilich MAEVSKY, Pushchino (RU); Vladimir Konstantinovich KLUBKOV, Moscow (RU)

(21) Appl. No.: 14/432,384

(22) PCT Filed: Oct. 2, 2012
(86) PCT No.: PCT/RU2012/000802

§ 371 Date: Mar. 30, 2015

(51) Int. Cl. C12N 13/00

(52) U.S. Cl. CPC C12N 13/00

(57) ABSTRACT

Method for activation of stem cells' proliferation and increase in the resistance of the stem cells to negative impacts, without use of high-frequency electro-magnetic field, is provided. The technical result is achieved due to the treating of the cells culture with a weak low-frequency magnetic field. The proposed method includes: 1) Increase in number of the human stem cells in the culture after their exposure to a weak magnetic field over a representative for the given cell culture doubling half-period (for 24 hours, number of cells increases 2.5 times-at full doubling period of the intact cells equals to 48 hours); 2) Increase of amplitude of self-magnetic irradiation of the initial culture of the stem cells, which indicates increased activity. 3) Double increase in stability of the human stem cells with respect to the development of apoptosis and synchronization of the cells predominantly in G1 phase.

### CROSS-REFERENCE TO RELATED APPLICATION

This Application is a US National Phase of PCT/RU2012/000802 filed on Oct. 2, 2012.

### BACKGROUND OF THE INVENTION

1. Field of the Invention

The invention is related to biotechnologies, medicine, pharmacology, cell biology and biological engineering and may be used in areas of biology and medicine using the stem cells and progenitor cells of various differential levels and post mitotic mature cells of various tissues and, in particular, for the rapid development of bio-implants from donors or autoimmune stem cells of humans and/or animals.

2. Description of the Related Art

Currently, there is a great number of technical solutions offering different methods for biophysical impact on the cells of plants and animals (RU Pat. No. 2,332,841 issued Sep. 10, 2008; RU Pat. No. 2,314,844 dated Jan. 20, 2008; RU Pat. No. 2,174,850 issued Oct. 20, 2001; RU Pat. No. 2,049,501 issued Dec. 10, 1995; RU Pat. No. 2,158,147 issued Oct. 27, 2000). The general drawbacks of all these inventions are in the impact duration (not less than 3 days), multi-staging of the methods and the impact on the biomaterial with extreme loads.

Another method is described in RU Pat. No. 2,405,599 (Dec. 10, 2010). This method includes irradiating of the bio object with the external electro-magnetic field with the measured parameters. The irradiation is performed in the living organism in the area of anatomic location of the red bone marrow with the electro-magnetic irradiation of the extremely high frequency of 35-80 GHz range with the surface density of the power flow of 0.1-10 mW/cm² range, amplitude-modulated with a modulation frequency variation within 4-10 Hz range. The flow density is within 0.1-10 mW/cm². The method provides for activation of development of the stem cells of the red bone marrow with the simultaneous stimulation of the processes of proliferation and differentiation of the red bone marrow cells in an organism.

The drawback of this invention is that the electro-magnetic irradiation of GHz frequencies has a dangerous impact on the biological objects, in particular, to those carrying the genetic information conserved in the nucleoproteins of the stem cells, which is strictly preserved as distinct feature from other cells.

Accordingly, an efficient method for activation of the stem cell proliferation and increase in the resistance to negative impacts of the stem cells of humans and animals without the negative impact of the high-frequency electro-magnetic field is desired.

### SUMMARY OF THE INVENTION

Accordingly, the present invention is related to a method for activation of the stem cell proliferation and increase in the resistance to negative impacts of the stem cells of humans and animals without the negative impact of the high-frequency electro-magnetic field that substantially obviates one or more of the disadvantages of the related art.

In one embodiment, a method for activation of the stem cells' proliferation and increase in the resistance of the stem cells of humans and animals to the negative impacts, without the negative impact of the high-frequency electro-magnetic field is provided. The technical result is achieved due to the treating of the cells culture with a weak low-frequency magnetic field. The proposed method includes: 1) Increase in the number of the human stem cells in the culture after their exposure to a weak magnetic field over a representative for the given cell culture doubling half-period (for example, for 24 hours, the number of cells increases more than 2.5 times-at full doubling period of the intact cells equals to 48 hours); 2) Increase of the amplitude of the self-magnetic irradiation of the initial culture of the stem cells, as measured by SQUID-type magnetometer, which indicates their increased activity. 3) Double increase in stability of the human stem cells with respect to the development of apoptosis and synchronization of the cells predominantly in the G1 phase of the cell cycle.

Additional features and advantages of the invention will be set forth in the description that follows, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BREIF DESCRIPTION OF THE ATTACHED FIGURES

The accompanying drawings, which are included to provide a further understanding of the invention and are incorporated in and constitute a part of this specification, illustrate embodiments of the invention and together with the description serve to explain the principles of the invention.

In the drawings:

FIGS. 1A and 1B illustrates the increase in the number of mesenchymal stem cells (MSCs) of a human after their irradiation with the alternating magnetic field, in accordance with the exemplary embodiment;

FIGS. 2A and 2B illustrate the results of the flow cytometry of the stem cells culture treated according to the suggested method, in accordance with the exemplary embodiment;

FIGS. 3A, 3B and 3C illustrate the results of electrophoresis and PCR products.

### DETAILED DESCRIPTION OF THE PREFERRED EMBODIMENTS

Reference will now be made in detail to the preferred embodiments of the present invention, examples of which are illustrated in the accompanying drawings.

The present invention is directed to a method for activation of the stem cells' proliferation and increase in the resistance of the stem cells of humans and animals to the negative impacts, without the negative impact of the high-frequency electro-magnetic field is provided. According to an exemplary embodiment, the technical result is achieved by the treatment of the cells culture with a weak low-frequency magnetic field, i.e., magnetic fields with frequencies between 7 and 72 Hz, and microTesla range (up to 100 microTesla). The inventive method uses the acting factor in a form of the weak alternating magnetic field collinear to the Earth's magnetic field. The inventors have discovered that orthogonal vectors leads to the formation of non-equivalent cells (i.e., an undesirable result). But the goal is the opposite-to obtain the maximum possible number of equivalent pool of stem cells. So, orthogonality results in spotty stem cell differentiation, 45° angle decreases the effect and a lack of co-directionality follows the negative effects and cell death. With a loss of collinearity, the entire spectrum of the unique genomic characteristics that was received in the collinear field would be. This is summarized below:

| Installation of the | (*)Caspase-3 activity, | Change in speed |
|---|---|---|
| resulting impact | nmol AFC/min/mkg of | of culture growth |
| of magnetic field | protein, after 24 hours | after 24 hours |
| Collinear to Earth | 0 | Increase by 2.8 times |
| magnetic field | | |
| Orthogonal to Earth | 1.500 ± 0.003 | Decrease by 1.5 times |
| magnetic field | | |
| 45° to the Earth | 0.340 ± 0.004 | cell growth was not |
| magnetic field vector | | observed |

Induction of caspase-3 which is the primary marker triggering Bax-dependent apoptosis. Thus, this shows a decrease of not only growth rate, but also increases in the pool apoptotic cells.
(*)Determination of caspase-3 in cultured cells was carried out by the method Barge R M Y, et al, (1997) Thornberry N A (1994).

The magnetic induction B and frequency f is determined by the formula proposed by Lednyev (V. V. Lednyev, Biological effects of the extremely weak alternating magnetic fields: identification of the initial targets, in “Modeling of geo-physical processes.” 2003, pages 130-136). The Lednev paper only proposed a theory mostly based on visual observations, without a strong theoretical background, and Lednev was not working with stem cells. Lednev et al. used a protractor for measurements, which has a high error. The inventors showed that this is an example of cooperative Jahn-Teller effect for complicated molecules (https:**books.google.ru/books?id=YX6JJR_yt1AC&pg=PA555&1pg=PA555&dq=cooper ative+Jahn%E2%80%93Teller+effect+for+complicated+molecules&source=b1&ots=k5_{—}4 gbyjKI&sig=AVj sbFf1A3kMrOCmIr-B1ZhbFSk&h1=ru&sa=X&ei=RIL1VLH1D-XjywOk6oLQAQ&ved=0CEcQ6AEwB g#v=onepage&q=cooperative%20Jahn%E2%80% 93Teller%20effect%20for%20complicated%20molecules&f=false):

B=B_{DC}+B_{AC} cos 2πft,

where B_{DC} and B_{AC}—are the values of the magnetic induction of the constant (of the Earth field) and variable component of the field (set by the magnetic generator) respectively, f—the frequency of the variable component. In addition, f frequencies of the alternating magnetic field, known as the resonance for the Ca²⁺ ion in a cell, where f is selected out of the range of f=25-42 Hz, with 37.1 Hz apparently optimal, based on experimental data.

The use of the low-frequency magnetic field does not produce the resonance and destructive properties and is comparable in terms of tension to the value of Earth magnetic field, and it is also collinear to Earth magnetic field. According to the exemplary embodiment, the stem cells of a human are irradiated under the suggested method providing for the activation of a number of intracellular processes, in particular, the polarization of the cell membrane, the change of the total dipole cell moment, the activation (acceleration) of the centrioles doubling process, as well as the change of the mitotic spindle orientation and structural and functional changes of the stem cell fate determinants (see E. V. Orlova, The role of structure-to-function particularities of cell fate determinants in mitotic spindles orientation and formation of niche complex, in Medline.ru, 2009, p. 10, p. 113-126). The set of these actions prepares the mesenchymal stem cells to the directed differentiation and accelerated proliferation in the set direction.

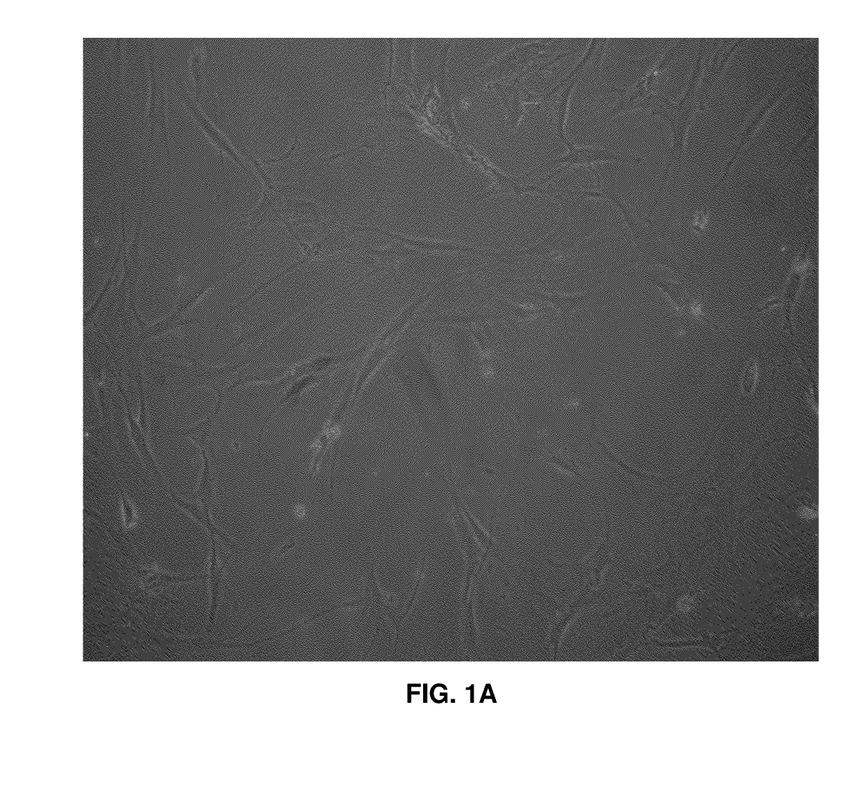

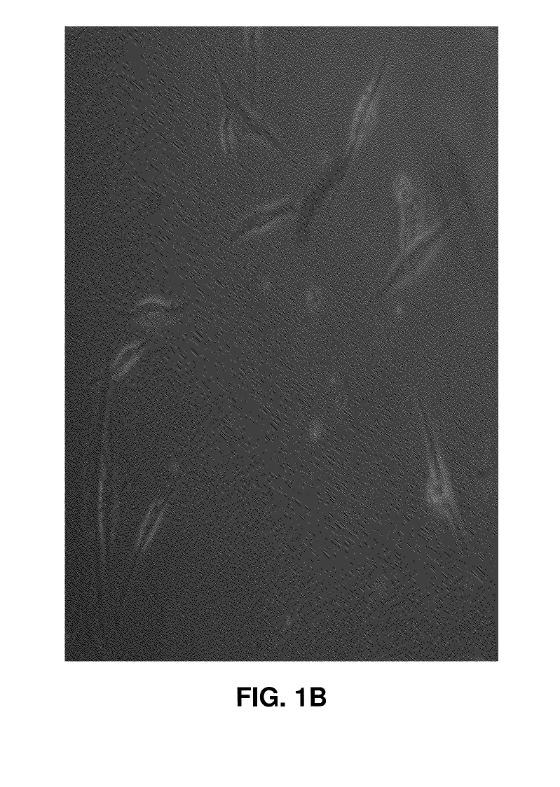

FIGS. 1A and 1B illustrate the increase in the number of mesenchymal stem cells (MSCs) of a human after their irradiation with the alternating magnetic field B_{AC}=89.4 μTl; frequency f=37.1 Hz; B_{DC}=48.6±0.1 μTl; B_{AC}=89.4 ±0.3 μTl; frequency f=37.1±0.1 Hz, temperature of the thermostatted cell −37° C. within 24 hours (e.g., 18 to 48, preferably around 20-30 hours, half of the standard period of doubling). In this particular experiment, the most preferred values were 37° C. and 24 hours.

FIG. 1A illustrates results from an oxidated environment with a light brown color (not seen in the picture), in comparison to the cells treated without the impact of the low-frequency magnetic field shown in FIG. 1B representing a conditioned environment with a standard color for α-MEM (Minimum Essential Medium Eagle Alpha Modifications, see http:**www.sigmaaldrich.com/life-science/cell-culture/classical-media-salts/mem-media.html).

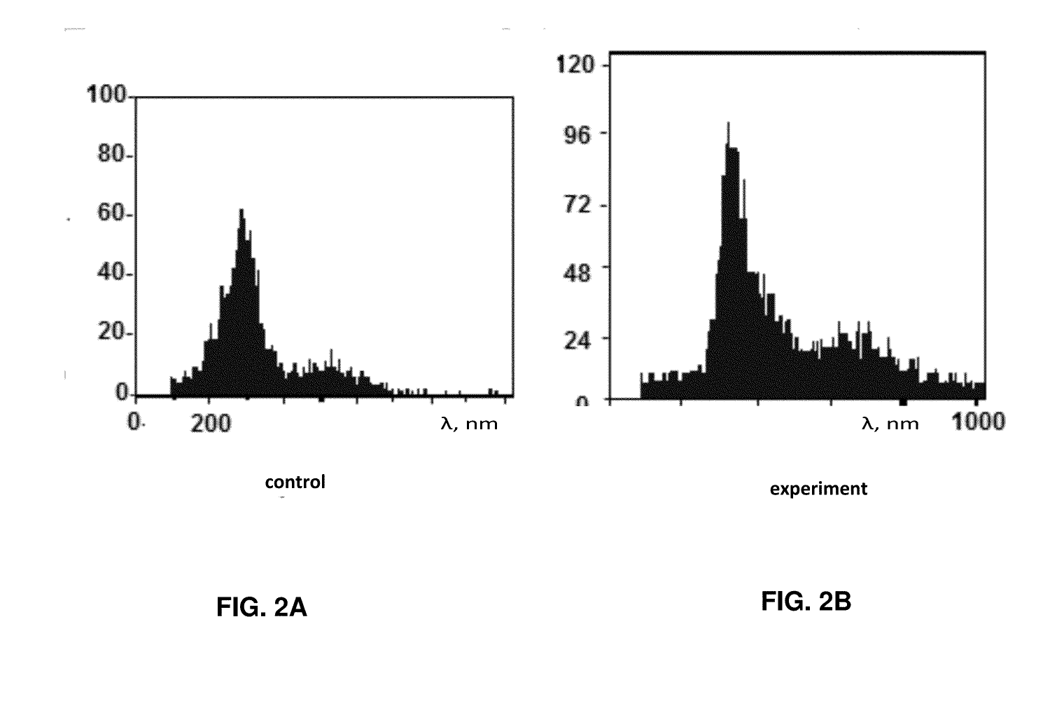

FIGS. 2A and 2B illustrate the results of the flow cytometry of the stem cells culture treated according to the suggested method. FIG. 2A illustrates a control specimen, FIG. 2B shows a culture after the treatment with the inventive method. As shown in these figures, activated cells undergo apoptosis much less than non-activated cells, and make a simultaneous transition of a cell cycle, growing more vigorously.

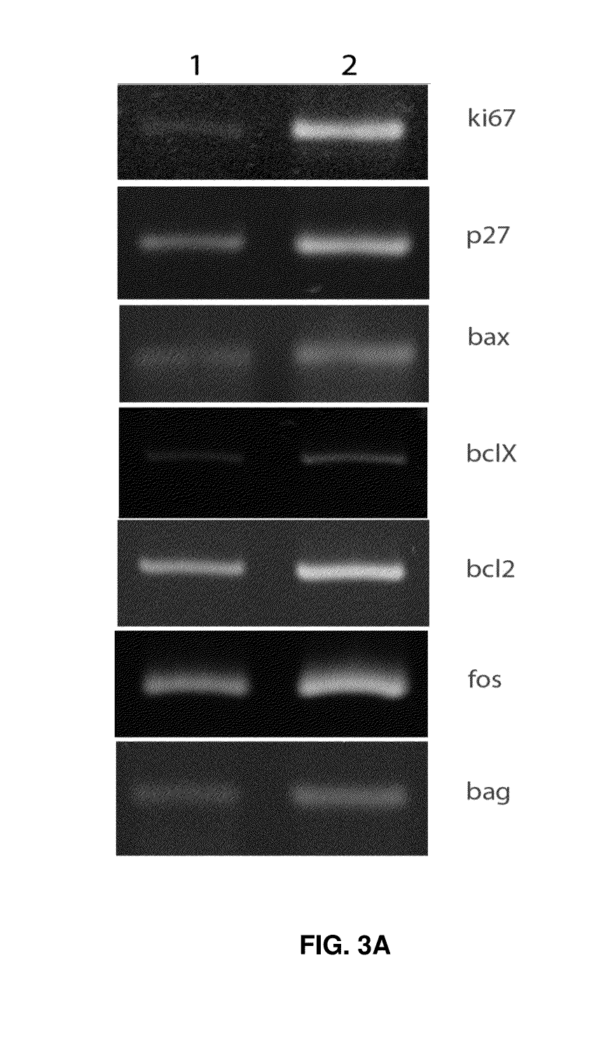

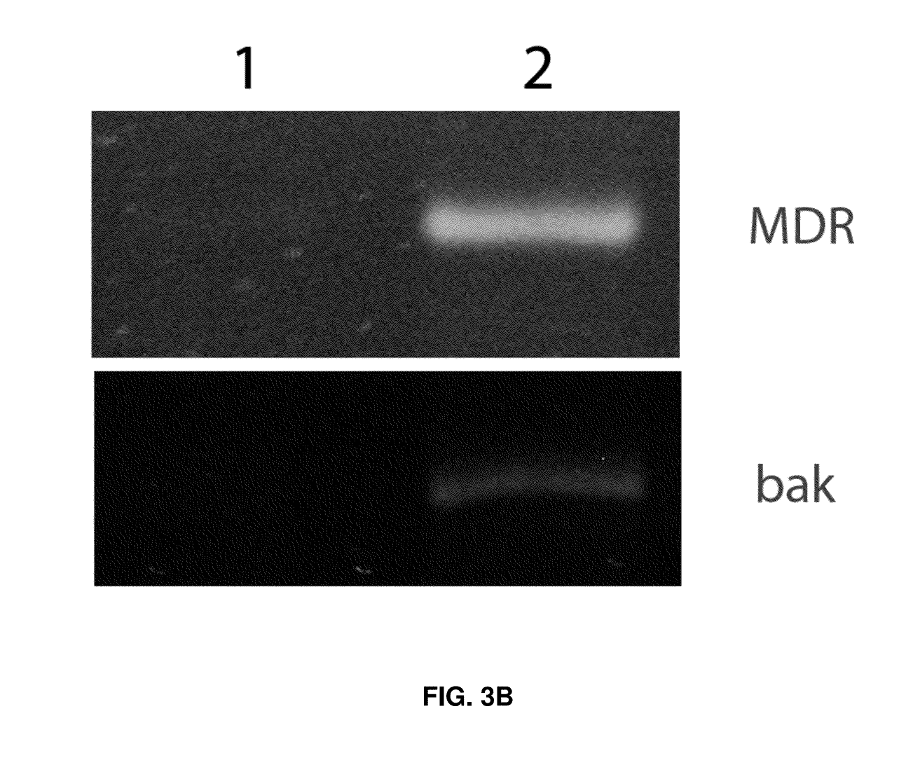

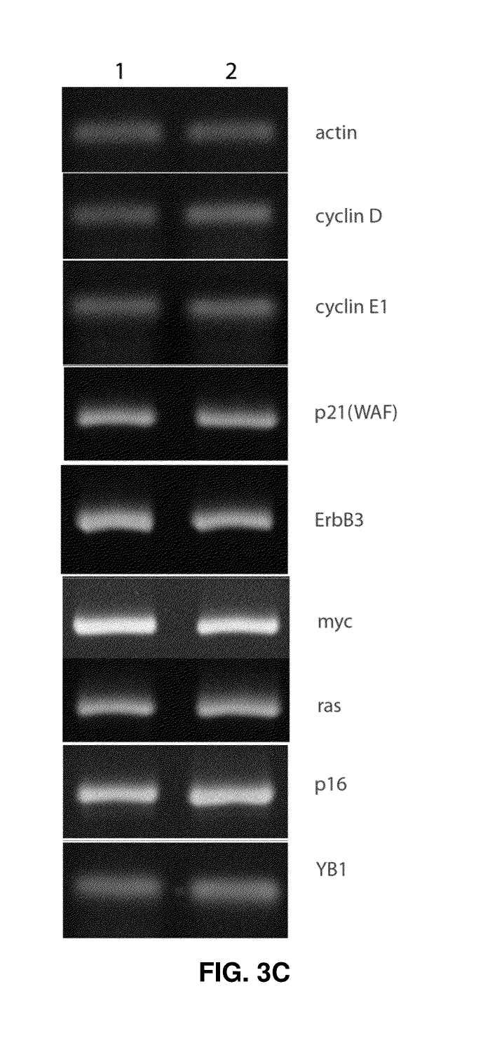

FIGS. 3A, 3B and 3C illustrate the results of electrophoresis and PCR products.

The exemplary method can be implemented as follows. The experiment was carried out on the mesenchymal stem cells (MSCs) isolated from human fat (“Biolot” company, Saint Petersburg, RU).

The doubling period of these cells is initially 48 hours. The cells were taken in the 3^{rd} passage and placed in the T₂₅ culture flasks with the environment of α-MEM (Modified Eagle's Medium) standard for these cells. The following experiments have been conducted: experiment and control, each used about 200,000 cells/ml.

During the experiment, the static magnetic field of the Earth is measured as B_{DC}=48.6 μTl; and the alternating magnetic field has been set with the help of the magnetizing system as B_{AC}=89.4 μTl; frequency f=37.1 Hz is collinear to the Earth field under the mentioned above formula: B_{DC}=48.6±0.1 μTl; B_{AC}=89.4±0.3 μTl; frequency f=37.1±0.1 Hz; B=B_{DC}+B_{AC} cos 2πft, where BAC [T1]—the amplitude of the external magnetic field; f [Hz]—its frequency; irradiation time—24 h, temperature of the thermostatted cell is 37° C.

In the above experiment, the cells were placed into the experimental cells under the extreme conditions with the increased density close to the maximal one for these cells. Under such conditions, the microscopy reveals the effect of reducing the number of the dendritic shoots and the cells are arranged into the columns, the appearance of the columns is the first visual evidence of the cells readiness to differentiate into the fibroblasts (i.e., there were created negative conditions, which increased the probability of losing the stem cells properties in the treated cells).

The 24 hours of exposure to the weak magnetic field (out of CO₂-incubator without stabilizing the pH environment) revealed the increase in the number of dendric shoots (extensions) of the cells before the basic level of the standard for the given culture of the stem cells, i.e., the maximum branching of the dendrites and the cells spreading were present even with the oxidized environment (FIG. 1A). Thus, the experiment featured the increase in the number of cells by 2.8 times, i.e. there was the acceleration of the culture growth (see FIGS. 1A, 1B), despite of the unsuitable cell treating conditions.

So, the cells treatment with the help of the low-frequency magnetic field in the selected range with the above described initial values not only accelerates the culture growth of the human stem cells by 2.8 times, but also minimizes the impact of the negative shifting of pH, thus supporting the proliferation and differentiation. In other words, the proposed system implements a condition for the selection of the system stability (enthalpy of the system/cell is minimized), and as the consequence the spontaneous disturbance (caused, for example, by the non-optimal treatment regime in the system) is also minimized. The increase of the amplitude of the self irradiation of the stem cells initial culture recorded by the superconducting quantum interference device (SQUID) testifies to the cells' increased activity.

According to the exemplary embodiment, the treated stem cells are less influenced by the apoptosis and conduct more synchronous transitions through the stages of the cell cycle, as shown in FIGS. 2A, 2B. The experiment has also analyzed the results of gene expression after activating of the proliferation in the alternating magnetic field during the halftime of doubling of the human stem cells characteristic of the cells culture. In the controlled and the experimental cells the following is compared: the level of mRNA for studying the gene expression participating in the cell cycle regulation, proliferation, differentiation processes, and cells death—cyclinD1 (the official symbol: CCND1), cyclinE1 (CCNE1), p21/waf (CDKN1A), ErbB3 (ERBB3), ki67 (MKI67), MDR1 (ABCB1), p16 (CDKN2A), p27/kip (CDKN1B), YB1 (YBX1), bax (BAX),bak (BAK1), bc1XL (BCL2L1), bc12 (BCL2), fos (FOS), myc (MYC), ras (HRAS1), bag (BAG1).

According to the exemplary embodiment, a total RNA isolation is analyzed. For the analysis, suspensions of control and experimental cells (˜10-12×10⁶) were used. The cells were centrifuged for 10 minutes at 4000 g. Cell pellets were re-suspended in 2 ml of lysis solution containing 4 M guanidine isothiocyanate, 0.02 M sodium citrate, 0.5% sarkosyl, 0.1 M mercaptoethanol.

Then, 1/10 of 1 M sodium acetate with pH of 4.4 has been added. After stifling, an equal volume of phenol equilibrated was added with water and 1.5 volume of chloroform and isoamyl alcohol (24:1). The mixture was vortexed and incubated for 15 minutes at +2-+8C.°. Next, the tubes had been centrifuged in the swinging bucket rotor for 20 minutes at 4000 g with cooling. The upper phase was transferred to another tube. It was added with an equal volume of phenol-chloroform (2:1), vortexed and centrifuged again for 20 minutes at 4000 g.

Using flow cytometry, an equal volume of isopropyl alcohol had been added to the upper phase and left overnight at −20 C.°. Then, it was centrifuged for 20 minutes at 4000 g, (pellet was washed with 80% ethanol and dissolved in 100 μL of water, treated with diethylpyrocarbonate). The RNA solution was added with 1/20 volume of 4 M LiCl and 2 volumes of ethanol. The isolated RNA was stored at −20 C. The concentration of the isolated RNA was measured with the spectrophotometer Smartspec plus (BioRad).

The reaction of the reverse transcription is analyzed as follows. The RNA pellet (10 mg) under the ethyl alcohol was washed with 1 ml of 80% ethanol and dissolved in 12 μL, of water. The test tube was added with 1 μL, of 10 mM oligodT and incubated at 70C° for 5 minutes. After cooling on ice for 5 minutes, the mixture was left at the room temperature for 15 minutes. Then, 4 μL 5× buffer solution was added for the reverse transcription, 2 μL, of 10 mM dNTP, 1 μL of reverse transcriptase Revert Aid (Fermentas). The reaction was carried out for one hour at 42C.°. The samples were stored at −20C.°.

The polymerase chain reaction occurs as follows:

The amplification of the studied genes was conducted in the mix of the following components:

10×Taq buffer(Fermentas)—2 μL;

25 mM MgCl₂—1.6 μL;

10 mM dNTP—0.4 μL;

10 mM primer 1-0.2 μL;

10 mM primer 2—0.2 μL;

Taq polymerase (5 U/μL) (Fermentas)—1 μL;

DNA (the product of the reverse transcription) 3 μL;

water—1.6 μL; and

in the amplifier MasterCycler gradient (Eppendorf) under the following conditions:

| Operations | Temperature ° C. | Time | | Number of cycles |
|---|---|---|---|---|
| Initial denaturation | 95 | 5 | min | 1 |
| Denaturation | 95 | 20 | sec | 40 |
| Annealing | 60 | 20 | sec | |
| Synthesis | 72 | 30 | sec | |
| Final synthesis | 72 | 2 | min | 1 |

The selection of the oligonucleotide primers and conditions for performing PCR was conducted by using the program Oligo 4.0. For performing PCR the following primers are were used:

CyclinD1
5′CTGCGAGGAACAGAAGTGCGAGG 3′
CyclinD2
5′GGATGGAGTTGTCGGTGTAGATGCA 3′
CyclinE1
5′ACCGTTTTTTTGCAGGATCCAGATG 3′
CyclinE2
5′GATGGTGCAATAATCCGAGGCTTG 3′
P211
5′CTTCGGCCCAGTGGACAGCG 3′
P212
5′CGTGGGAAGGTAGAGCTTGGGC 3′
ErbB1
5′CCTGAGTGTGACCGGCGATGC 3′
ErB2
5′AGAGAATTCATTCATGGCCACGAGG 3′
Ki671
5′TGTGACATCCGTATCCAGCTTCCTG 3′
Ki672
5′CATTTTCATACCTGAAGGAACGATCAATAA 3′
MDR1
5′TTTCAATGTTTCGCTATTCAAATTGGC 3′
MDR2
5′GTTTGACATCAGATCTTCTAAATTTCCTGC 3′
P161
5′CCCTGGAGGCGGCGAGAAC 3′
P162
5′CCTAGACGCTGGCTCCTCAGTAGC 3′
P271
5′CCGGGACTTGGAGAAGCACTGC 3′
P272
5′GGCACCTTGCAGGCACCTTTG 3′
YB1
5′TCCCACCTTACTACATGCGGAGACC 3′
YB2
5′TAGGCTGTCTTTGGCGAGGAGG 3′
Bc121
5′GCCCTGTGGATGACTGAGTACCTGAAC 3′
Bc122
5′GCCAAACTGAGCAGAGTCTTCAGAGACA 3′
Bax1
5′TTAGGATCCGGGAGCAGCCCAGAG 3′
Bax2
5′TTAAGCTTGACCTCTCGGGGGGAGTC 3′
Bak1
5′ATAGGATCCTGGCTTCGGGGCAAGG 3′
Bak2
5′GAGAAGCTTGTACTCATAGGCATTCTCTGCCG 3′
BclX1
5′TATGGATCCAGCTTTCCCAGAAAGGATACAG 3′
BclX2
5′CGGAAGCTTGCTCTGATATGCTGTCCC 3′
Bag1
5′ATCCCTGGCCTTCATCAG 3′
Bag2
5′GCACTGCTAGGCCATGG 3′
Fos1
5′AGATGTCTGTGGCTTCCCTTGATCTG 3′
Fos2
5′AAGTCATCAAAGGGCTCGGTCTTCA 3′
Myc1
5′AACAATGAAAAGGCCCCCAAGGTA 3′
Myc2
5′TCCGTAGCTGTTCAAGTTTGTGTTTCAA 3′
Ras1
5′GACGAATATGACCCCACAATAGAGGATTC 3′
Ras2
5′ATTATTGATGGCAAATACACACAGGAAGC 3′

To confirm the equal quantities of nucleic acids in the samples we used the amplification of the actin gene with the relevant primers:

Actin1
CCAACACAGTGCTGTCTGGCGG
Actin2
TACTCCTGCTTGCTGATCCACATCTG

According to one exemplary embodiment, electrophoresis and quantification of PCR products is implemented. The products of the PCR reaction were separated in the 6% polyacrylamide gel (composition: 1.4 ml of a 30% solution AA 1.4 ml 5× TBE buffer, 4.2 ml dist. water, 30 μLof the 10% ammonium persulfate (APS), 20 μLof TEMED.) in 1× TBE buffer (0.089 M Tris, 0.089 M of boric acid, 0.002 M of EDTA pH 8.3) for 40 minutes at 20 mA. After staining the solution with the ethidium bromide (1 mg/ml) the gels were photographed and measured using TotalLab V2.01.v in comparison with the standard samples.

For this purpose, the standard samples with the known concentration of the complementary DNA were tittered on the polyacrylamide gel. After scanning the gel, the intensity of the bands was quantitatively measured using the program. Then, a calibration graph that defines the relative amounts of the studied amplicons is drawn. The quantification of the results shown in FIG. 3A in relation to the control (1) in the experimental cells (2) illustrates the change in the amount of mRNA of the following genes:

ki67-4× increase;

p27-2× increase;

bax-1.5× increase;

bclX-1.5× increase;

bc12-2× increase;

fos-2× increase;

bag-1.7× increase.

Thus, the activation of the antiapoptotic genes is more evident than the pro-apoptotic ones (bax), but one must take into account that the apoptotic proteins Bax and Bak can form stable blocking the apoptosis conglomerates with the antiapoptotic proteins (for example, BclX and Bc12).

Furthermore, in the experimental cells the de novo synthesis of mRNA MDR (multi drug resistant) gene and bak are shown (see FIG. 3B). The increased expression of the MDR is connected with drug resistance of the mammalian cell cultures (see Croop J M, 1993, “P-glycoprotein structure and evolutionary homologies.” Cytotechnology 12: 1-32).

At that, the genes expression of cyclinD, cyclinE, p21 (WAF), ErbB3, p16, YB1, myc, ras remained unchanged (see FIG. 3B) that indicates the absence of the ability of the activated stem cells for transformation into the tumor ones (if there is such a possibility, the activation of the above series of cyclins occurs).

Therefore, the achievement of the technical result is proved as follows:

1. Increase in the number of the human stem cells in the culture after their exposure in a weak magnetic field over a representative for the given cell culture doubling halfperiod (for instance, for 24 hours), the number of cells increases more than 2.5 times (at full doubling period of the taken intact cells equals to 48 hours);

2. Increase of the amplitude of the self-magnetic irradiation of the initial culture of the stem cells, as measured by SQUID-type magnetometer, which indicates their increased activity.

The human stem cells, after their exposure to a weak magnetic field over a representative for the given cell culture doubling half period, are 2 times more stable with respect to the development of apoptosis and are synchronized predominantly in the G1 phase of the cell cycle.

Having thus described a preferred embodiment, it should be apparent to those skilled in the art that certain advantages of the described method and apparatus have been achieved.

It should also be appreciated that various modifications, adaptations, and alternative embodiments thereof may be made within the scope and spirit of the present invention. The invention is further defined by the following claims.

What is claimed is:

1. A method for treating stem cells by a magnetic field, the method comprising:
exposing the stem cells to an alternating magnetic field, wherein:
the magnetic field is applied collinearly to Earth magnetic field; and
the magnetic field activates proliferation and increases resistance of the stem cells to negative impacts that decrease normal metabolism, homeostasis, proliferation, differentiation and growth of stem cells.

2. The method of claim 1, wherein a frequency of the alternating magnetic field is within a range from 25 to 42 Hz.

3. The method of claim 1, wherein a magnitude of the alternating magnetic field is comparable to a magnitude of the Earth magnetic field and its amplitude is within the range from 75 to 110 μTl.

4. The method of claim 1, wherein irradiation of the cells under the alternating magnetic field provides for activation of any of intercellular processes:
polarization of cell membrane;
change of a total dipole cell moment;
acceleration of centrioles doubling process;
change of mitotic spindle orientation; and
functional changes of the stem cell fate determinants.
